(19) United States
(12) Patent Application Publication
(10) Pub. No.: US 2021/0386406 A1
(43) Pub. Date: Dec. 16, 2021

(54) ULTRASOUND DIAGNOSTIC APPARATUS, MEDICAL IMAGE PROCESSING APPARATUS, AND MEDICAL IMAGE PROCESSING METHOD

(71) Applicant: CANON MEDICAL SYSTEMS CORPORATION, Otawara-shi (JP)

(72) Inventor: Yasuhiko ABE, Otawara (JP)

(73) Assignee: CANON MEDICAL SYSTEMS CORPORATION, Otawara-shi (JP)

(21) Appl. No.: 17/343,907

(22) Filed: Jun. 10, 2021

(30) Foreign Application Priority Data
Jun. 11, 2020 (JP) 2020-101624

(51) Int. Cl. A61B 8/08; A61B 8/02

(52) U.S. Cl. CPC A61B 8/5207; A61B 8/02

(57) ABSTRACT

An ultrasound diagnostic apparatus according to embodiments includes processing circuitry. The processing circuitry acquires a plurality of pieces of medical image data arranged in time series over at least one cardiac cycle in which a region including a pulsative target of a subject is imaged. The processing circuitry performs a plurality of motion estimation processes using a pattern matching at frame intervals different from each other on an identical position for the pieces of medical image data and determines most likely second motion information from among a plurality of pieces of first motion information estimated by the motion estimation processes.

### CROSS-REFERENCE TO RELATED APPLICATIONS

This application is based upon and claims the benefit of priority from Japanese Patent Application No. 2020-101624, filed on Jun. 11, 2020; the entire contents of which are incorporated herein by reference.

### FIELD

Embodiments described herein relate generally to an ultrasound diagnostic apparatus, a medical image processing apparatus, and a medical image processing method.

### BACKGROUND

In echocardiography using an ultrasound diagnostic apparatus, cardiac function evaluation is performed which measures and estimates the shape of myocardium from the captured two-dimensional or three-dimensional image data and calculates a variety of cardiac function indices. In cardiac function evaluation, for example, the modified-Simpson's method that estimates a three-dimensional shape of myocardium from the contour shape of myocardium in two different sections is used. In the modified-Simpson's method, an apical four-chamber view (A4C) and an apical two-chamber view (A2C) are used as two sections, for example. Then, the three-dimensional shape of myocardium is estimated from the contour shapes of myocardium visualized in two sections, whereby volume information such as end diastolic volume (EDV), end systolic volume (ESV), and ejection fraction (EF) of left ventricle (LV) and global longitudinal strain (GLS) information are calculated as global cardiac function indices. The acquisition of EF and GLS information is implemented, for example, in applications using speckle-tracking echocardiography (STE).

STE is applicable not only to two-dimensional image data but also to three-dimensional image data. STE can be applied to three-dimensional image data to analyze cardiac functions, whereby the three-dimensional shape of myocardium can be three-dimensionally measured and EF and GLS information can be calculated based on the measurement result.

### BRIEF DESCRIPTION OF THE DRAWINGS

FIG. 1 is a block diagram illustrating a configuration example of an ultrasound diagnostic apparatus according to a first embodiment;

FIG. 2 is a diagram for explaining the basic principle of motion estimation according to the first embodiment;

FIG. 3 is a flowchart illustrating a procedure in the ultrasound diagnostic apparatus according to the first embodiment;

FIG. 4 is a flowchart illustrating a procedure in the ultrasound diagnostic apparatus according to the first embodiment;

FIG. 5 is a diagram for explaining a process of a tracking function according to the first embodiment;

FIG. 6 is a flowchart illustrating a procedure in the ultrasound diagnostic apparatus according to a first modification to the first embodiment;

FIG. 7 is a diagram for explaining a process of the tracking function according to a second modification to the first embodiment;

FIG. 8 is a flowchart illustrating a procedure in the ultrasound diagnostic apparatus according to a second embodiment;

FIG. 9 is a diagram for explaining a process of the tracking function according to the second embodiment; and

FIG. 10 is a block diagram illustrating a configuration example of a medical image processing apparatus according to other embodiments.

### DETAILED DESCRIPTION

An ultrasound diagnostic apparatus according to embodiments includes processing circuitry. The processing circuitry acquires a plurality of pieces of medical image data arranged in time series over at least one cardiac cycle in which a region including a pulsative target of a subject is imaged. The processing circuitry performs a plurality of motion estimation processes using a pattern matching at frame intervals different from each other on an identical position for the pieces of medical image data and determines most likely second motion information from among a plurality of pieces of first motion information estimated by the motion estimation processes.

An ultrasound diagnostic apparatus, a medical image processing apparatus, and a medical image processing program according to embodiments will be described below with reference to the drawings. It should be noted that embodiments are not limited to the following embodiments. A description of one embodiment is basically applicable similarly to other embodiments.

### First Embodiment

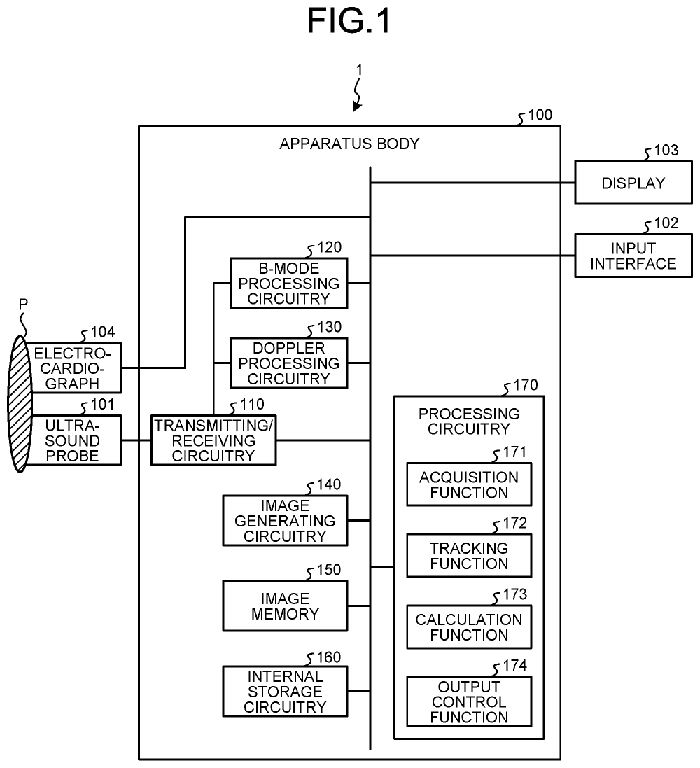

First of all, a configuration of an ultrasound diagnostic apparatus according to a first embodiment will be described. FIG. 1 is a block diagram illustrating a configuration example of an ultrasound diagnostic apparatus 1 according to the first embodiment. As illustrated in FIG. 1, the ultrasound diagnostic apparatus 1 according to the first embodiment includes an apparatus body 100, an ultrasound probe 101, an input interface 102, a display 103, and an electrocardiograph 104. The ultrasound probe 101, the input interface 102, the display 103, and the electrocardiograph 104 are connected to communicate with the apparatus body 100.

The ultrasound probe 101 has a plurality of transducer elements, and the transducer elements generate ultrasound based on a driving signal supplied from transmitting/receiving circuitry 110 included in the apparatus body 100. The ultrasound probe 101 also receives a reflected wave from a subject P and converts the reflected wave into an electrical signal. The ultrasound probe 101 includes a matching layer provided to the transducer elements and a backing member for preventing propagation of ultrasound backward from the transducer elements. The ultrasound probe 101 is removably connected to the apparatus body 100.

When ultrasound is transmitted from the ultrasound probe 101 to the subject P, the transmitted ultrasound is reflected one after another on a discontinuous acoustic impedance surface of body tissues of the subject P and received as reflected wave signals by the transducer elements of the ultrasound probe 101. The amplitudes of the received reflected wave signals are dependent on an acoustic impedance difference in the discontinuous surface on which the ultrasound is reflected. When the transmitted ultrasound pulse is reflected on blood flow or a cardiac wall surface, for example, the reflected wave signal undergoes a frequency shift due to the Doppler effect, depending on a velocity component of the moving body with respect to the ultrasound transmission direction.

The input interface 102 includes, for example, a mouse, a keyboard, a button, a panel switch, a touch command screen, a foot switch, a trackball, and a joystick, and accepts a variety of setting requests from the operator of the ultrasound diagnostic apparatus 1 and transfers the accepted setting requests to the apparatus body 100.

The display 103 displays graphical user interfaces (GUIs) for the operator of the ultrasound diagnostic apparatus 1 to input a variety of setting requests using the input interface 102 or displays ultrasound image data and the like generated by the apparatus body 100. The display 103 also displays a variety of messages to notify the operator of a process status of the apparatus body 100. The display 103 may have a speaker to output sound. For example, the speaker of the display 103 outputs predetermined sound such as a beep to notify the operator of a process status of the apparatus body 100.

The electrocardiograph 104 acquires an electrocardiogram (ECG) of the subject P as a biological signal of the subject P. The electrocardiograph 104 transmits the acquired electrocardiogram to the apparatus body 100. In the present embodiment, the electrocardiograph 104 is used as one of means for acquiring information on cardiac phases of the heart of the subject P. However, embodiments are not limited thereto. For example, the ultrasound diagnostic apparatus 1 may acquire information on cardiac phases of the heart of the subject P by acquiring the time of the II sound (second sound) in a phonocardiogram or the aortic valve close (AVC) time obtained by measuring the outflow of the heart with spectrum Doppler.

The apparatus body 100 is an apparatus that generates ultrasound image data based on the reflected wave signals received by the ultrasound probe 101. The apparatus body 100 illustrated in FIG. 1 is an apparatus that can generate two-dimensional ultrasound image data based on two-dimensional reflected wave data received by the ultrasound probe 101. The apparatus body 100 is an apparatus that can also generate three-dimensional ultrasound image data based on three-dimensional reflected wave data received by the ultrasound probe 101.

As illustrated in FIG. 1, the apparatus body 100 includes transmitting/receiving circuitry 110, B-mode processing circuitry 120, Doppler processing circuitry 130, image generating circuitry 140, an image memory 150, internal storage circuitry 160, and processing circuitry 170. The transmitting/receiving circuitry 110, the B-mode processing circuitry 120, the Doppler processing circuitry 130, the image generating circuitry 140, the image memory 150, the internal storage circuitry 160, and the processing circuitry 170 are connected to communicate with each other.

The transmitting/receiving circuitry 110 includes a pulse generator, a transmission delaying unit, and a pulser and supplies a driving signal to the ultrasound probe 101. The pulse generator repeatedly generates rate pulses for forming transmission ultrasound at a predetermined rate frequency. The transmission delaying unit converges the ultrasound produced from the ultrasound probe 101 into a beam and applies a delay time for each transducer element necessary for determining transmission directivity to the corresponding rate pulse generated by the pulse generator. The pulser applies a driving signal (driving pulse) to the ultrasound probe 101 at a timing based on the rate pulse. In other words, the transmission delaying unit adjusts the transmission direction of ultrasound transmitted from the transducer element surface as appropriate, by changing the delay time to be applied to each rate pulse

The transmitting/receiving circuitry 110 has a function that can instantaneously change a transmission frequency, a transmission driving voltage, and the like to execute a predetermined scan sequence based on an instruction from the processing circuitry 170 described later. Specifically, the transmission driving voltage can be changed by linear amplifier-type oscillator circuitry that can instantaneously switch its value or by a mechanism that electrically switches a plurality of power supply units.

The transmitting/receiving circuitry 110 includes a preamplifier, an analog-digital (A/D) converter, a reception delaying unit, and an adder and performs a variety of processes for the reflected wave signals received by the ultrasound probe 101 to generate reflected wave data. The preamplifier amplifies the reflected wave signal for each channel. The A/D converter performs A/D conversion of the amplified reflected wave signal. The reception delaying unit applies a delay time necessary to determine reception directivity. The adder performs an addition process for the reflected wave signal processed by the reception delaying unit to generate reflected wave data. As a result of the addition process by the adder, a reflection component from the direction corresponding to reception directivity of the reflected wave signal is emphasized, and a comprehensive beam of ultrasound transmission/reception is formed with reception directivity and transmission directivity.

Here, the output signal from the transmitting/receiving circuitry 110 may be selected from a variety of types, such as a signal including phase information called a radio frequency (RF) signal or amplitude information after an envelope detection process.

The B-mode processing circuitry 120 receives reflected wave data from the transmitting/receiving circuitry 110 and performs processes such as logarithm amplification and the envelope detection process to generate data (B-mode data) in which signal intensities are represented by brightness of luminance.

The Doppler processing circuitry 130 performs frequency analysis of velocity information from the reflected wave data received from the transmitting/receiving circuitry 110, extracts blood flow, tissues, and contrast agent echo components using the Doppler effect, and generates data (Doppler data) that is moving body information such as velocity, variance, and power extracted at multiple points.

The B-mode processing circuitry 120 and the Doppler processing circuitry 130 illustrated in FIG. 1 can process both of two-dimensional reflected wave data and three-dimensional reflected wave data. More specifically, the B-mode processing circuitry 120 generates two-dimensional B-mode data from two-dimensional reflected wave data and generates three-dimensional B-mode data from three-dimensional reflected wave data. The Doppler processing circuitry 130 generates two-dimensional Doppler data from two-dimensional reflected wave data and generates three-dimensional Doppler data from three-dimensional reflected wave data.

The image generating circuitry 140 generates ultrasound image data from data generated by the B-mode processing circuitry 120 and the Doppler processing circuitry 130. More specifically, the image generating circuitry 140 generates two-dimensional B-mode image data representing the intensities of reflected waves by luminance from the two-dimensional B-mode data generated by the B-mode processing circuitry 120. The image generating circuitry 140 also generates two-dimensional Doppler image data representing moving body information from the two-dimensional Doppler data generated by the Doppler processing circuitry 130. The two-dimensional Doppler image data is a velocity image, a variant image, a power image, or a combination image of these images. The image generating circuitry 140 can also generate M-mode image data from time-series data of B-mode data on a scan line generated by the B-mode processing circuitry 120. The image generating circuitry 140 can also generate Doppler waveforms in which velocity information of blood flow and tissues is plotted in time series, from the Doppler data generated by the Doppler processing circuitry 130.

The image generating circuitry 140 typically converts (scan converts) a sequence of scan line signals by ultrasound scanning into a sequence of scan line signals in a video format typically for televisions to generate ultrasound image data for display. Specifically, the image generating circuitry 140 generates ultrasound image data for display by performing coordinate conversion based on the scanning mode of ultrasound by the ultrasound probe 101. The image generating circuitry 140 also performs a variety of image processing other than scan conversion, such as image processing (smoothing process) of regenerating an image with average values of luminance using a plurality of image frames after scan conversion and image processing (edge enhancement process) using a differential filter in an image. The image generating circuitry 140 also combines character information of a variety of parameters, scales, and body marks with the ultrasound image data.

In other words, B-mode data and Doppler data are ultrasound image data before the scan conversion process, and data generated by the image generating circuitry 140 is ultrasound image data for display after the scan conversion process. B-mode data and Doppler data may be referred to as raw data. The image generating circuitry 140 generates “two-dimensional B-mode image data or two-dimensional Doppler image data” that is two-dimensional ultrasound image data for display, from “two-dimensional B-mode data or two-dimensional Doppler data” that is two-dimensional ultrasound image data before the scan conversion process.

The image memory 150 is a memory that stores image data for display generated by the image generating circuitry 140. The image memory 150 can also store data generated by the B-mode processing circuitry 120 or the Doppler processing circuitry 130. The B-mode data or the Doppler data stored in the image memory 150 can be invoked by, for example, the operator after diagnosis and passed through the image generating circuitry 140 to serve as ultrasound image data for display.

The image generating circuitry 140 stores ultrasound image data and the time of ultrasound scanning performed to generate the ultrasound image data in the image memory 150 in association with the electrocardiogram transmitted from the electrocardiograph 104. The processing circuitry 170 described later can refer to data stored in the image memory 150 to acquire the cardiac phases at the time of ultrasound scanning performed to generate ultrasound image data. The internal storage circuitry 160 stores a control program for performing ultrasound transmission/reception, image processing, and display processing, diagnosis information (for example, patient ID, doctor's finding), and a variety of data such as diagnosis protocols and body marks. The internal storage circuitry 160 can also be used for keeping image data stored in the image memory 150, if necessary. The data stored in the internal storage circuitry 160 can be transferred to an external device via a not-illustrated interface. The external device is, for example, a personal computer (PC), a storage medium such as CD or DVD, or a printer used by the doctor performing image diagnosis.

The processing circuitry 170 controls all the processes in the ultrasound diagnostic apparatus 1. Specifically, the processing circuitry 170 controls the processing in the transmitting/receiving circuitry 110, the B-mode processing circuitry 120, the Doppler processing circuitry 130, and the image generating circuitry 140, based on a variety of setting requests input by the operator through the input interface 102, and a variety of control programs and a variety of data read from the internal storage circuitry 160. The processing circuitry 170 also performs control such that ultrasound image data for display stored in the image memory 150 or the internal storage circuitry 160 appears on the display 103.

The processing circuitry 170 also performs an acquisition function 171, a tracking function 172, a calculation function 173, and an output control function 174. The acquisition function 171 is an example of an acquisition unit. The tracking function 172 is an example of a tracking unit. The calculation function 173 is an example of a calculation unit. The output control function 174 is an example of an output control unit. The processing of the acquisition function 171, the tracking function 172, the calculation function 173, and the output control function 174 performed by the processing circuitry 170 will be described later.

For example, the processing functions performed by the acquisition function 171, the tracking function 172, the calculation function 173, and the output control function 174, which are components of the processing circuitry 170 illustrated in FIG. 1, are stored in the internal storage circuitry 160 in the form of a computer-executable program. The processing circuitry 170 is a processor that reads and executes a computer program from the internal storage circuitry 160 to implement a function corresponding to the computer program. In other words, in a state in which a computer program is read out, the processing circuitry 170 has the corresponding function indicated in the processing circuitry 170 in FIG. 1.

In the present embodiment, the processing functions described later are implemented in the single processing circuitry 170. However, a plurality of independent processors may be combined to configure processing circuitry, and the processors may execute computer programs to implement the functions.

The word “processor” used in the description above means, for example, a central processing unit (CPU), a graphics processing unit (GPU), or circuitry such as an application specific integrated circuit (ASIC), a programmable logic device (for example, simple programmable logic device (SPLD), a complex programmable logic device (CPLD), and a field programmable gate array (FPGA). The processor reads and executes a computer program stored in the internal storage circuitry 160 to implement a function. A computer program may be directly embedded in circuitry in the processor, rather than storing a computer program in the internal storage circuitry 160. In this case, the processor reads and executes the computer program embedded in the circuitry to implement a function. The processors in the present embodiment are not limited to a configuration in which single circuitry is configured for each processor, but a plurality of pieces of independent circuitry may be combined into one processor and implement the function. Furthermore, a plurality of components in the drawings may be integrated into one processor to implement the function.

In speckle-tracking echocardiography (STE), myocardium is tracked by estimating motion (movement vector) at each position (each point) by the technique of pattern matching between frames. In a pattern matching process of comparing and searching for similar parts between images, in principle, motion is estimated only in units of one pixel (called “pixel” in two-dimensional images or “voxel” in three-dimensional images but, for the sake of simplicity, referred to as “pixel” for both images). For example, when one pixel is 0.3 mm, motion is unable to be estimated with accuracy smaller than this.

Then, a technique called subpixel estimation is used in combination to obtain motion components smaller than one pixel. Specifically, optical flow using the luminance gradient of a target changing with motion and subpixel estimation using response surface methodology for a spatial distribution of motion estimation index values are known. In STE, motion estimation is performed using an image having a speckle pattern of ultrasound. It is common to use the sum of squared difference (SSD) or the sum of absolute difference (SAD) as a motion estimation index value and perform subpixel estimation of motion components by response surface methodology that spatially interpolates the peak position of an index value distribution in the neighborhood of a position (denoted as “Pc”) where a movement vector in units of pixels is obtained.

The interpolated peak position is exactly on a pixel if the index value distribution is spatially symmetric with respect to Pc, but it deviates from a pixel if the distribution is asymmetric, and the degree of deviation is calculated. However, since there are limitations in spatial resolution of ultrasound beams (peak detection is failed in a dull index value distribution), the accuracy of subpixel estimation has limitations.

In order to perform accurate motion estimation for deformable myocardium, it is advantageous to reduce the amount of change of signals matched between frames (to increase the correlation between signals) by setting higher frame rates (frames per second (fps)).

On the other hand, the higher the frame rate is, the smaller the amount of motion of myocardium between frames is. Slow motion therefore is unable to be detected if an excessively high frame rate is set due to limitations in subpixel estimation. Under these requirements, in two-dimensional spectral tracking, ideal frame rates of 40 to 80 [Hz] are widely used in the range of normal cardiac rates (Non Patent Literature 1: Voigt JU et al, “Definitions for a common standard for 2D speckle tracking echocardiography: consensus document of the EACVI/ASE/Industry Task Force to standardize deformation imaging.” J Am Soc Echocardiography 28:183-93,2015).

With this, the accuracy in slow motion estimation may deteriorate and tracking may be failed in a circumstance that requires acquisition of moving images at a frame rate as high as over 100 [Hz], for example, when STE is applied to a fetal heart having a cardiac rate of about 150 [bpm], more than twice that of adults.

Even in STE application to an adult heart, since there are cardiac phases during which motion stops, such as end-systole and mid-diastole, motion sometimes fails to be detected with high accuracy at one-frame intervals, in such cardiac phases and myocardial parts having a low velocity. Consequently, the output values of EF and GLS information are underestimated. Moreover, this influence increases as the acquired image has a higher frame rate. The ultrasound diagnostic apparatus 1 according to the present embodiment then performs the processing functions described below in order to improve the accuracy in cardiac function evaluation. More specifically, in cardiac function evaluation using STE, the ultrasound diagnostic apparatus 1 enables highly accurate cardiac function evaluation by estimating a slow motion component with high accuracy even when the frame rate is high.

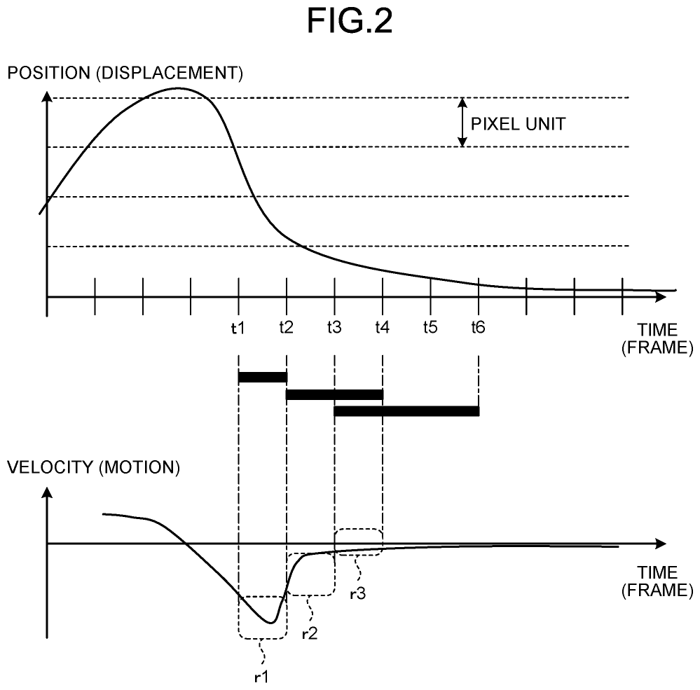

FIG. 2 is a diagram for explaining the basic principle of motion estimation according to the first embodiment. The basic principle described with reference to FIG. 2 is only an example and the present embodiment is not limited to the illustration in the drawing.

In the upper section in FIG. 2, the vertical axis corresponds to position (displacement) and the horizontal axis corresponds to time (frame). In the upper section in FIG. 2, each mark on the scale in the vertical axis corresponds to one pixel. In the lower section in FIG. 2, the vertical axis corresponds to velocity (motion) and the horizontal axis corresponds to time (frame). The horizontal axes (time axes) in the upper section in FIG. 2 and the lower section in FIG. 2 correspond to each other.

As illustrated in FIG. 2, the basic principle is that motion is estimated without decimation when a displacement is large (velocity is high) with respect to frame intervals, and motion is estimated with decimated frame intervals when a displacement is small (velocity is low). For example, the motion of a region r1 having a high velocity is estimated by the pattern matching process at one-frame intervals (image data at time t1 and time t2) without decimating images (frames). The motion of a region r2 having an intermediate velocity is estimated by the pattern matching process at two-frame intervals (image data at time t2 and time t4) by decimating one frame. The motion of a region r3 having a low velocity is estimated by the pattern matching process at three-frame intervals (image data at time t3 and time t6) by decimating two frames. The black bars depicted between the upper section in FIG. 2 and the lower section in FIG. 2 represent frame intervals for use in the pattern matching process.

In other words, the ultrasound diagnostic apparatus 1 according to the first embodiment improves the accuracy in cardiac function evaluation by executing the processing functions described below to automatically apply appropriate frame intervals (decimation intervals) depending on the velocity of a pulsative target. The processing functions will be described below.

In the following embodiment, STE is applied to two-dimensional image data (A4C image and A2C image). However, the present embodiment is not limited thereto. In other words, the present embodiment is applicable to STE for three-dimensional image data.

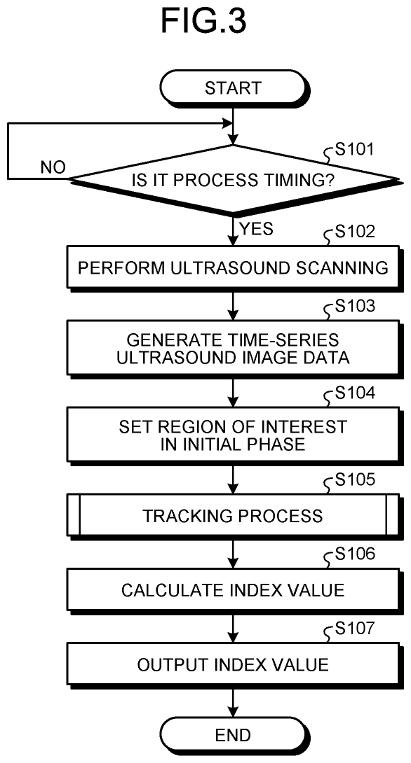

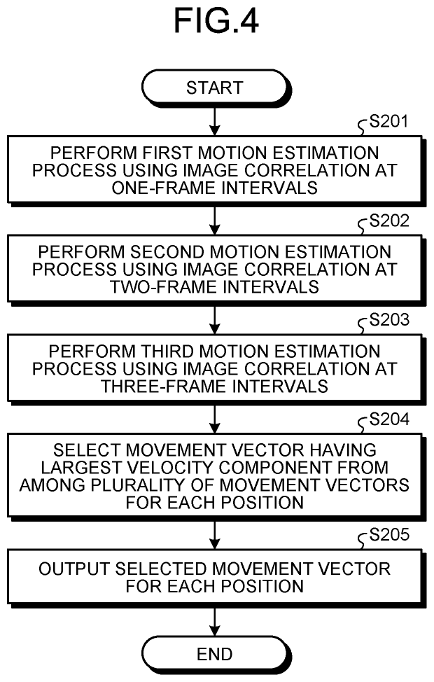

Referring to FIG. 3 and FIG. 4, a procedure in the ultrasound diagnostic apparatus 1 according to the first embodiment will be described. FIG. 3 and FIG. 4 are flowcharts illustrating the procedure in the ultrasound diagnostic apparatus 1 according to the first embodiment. The procedure illustrated in FIG. 3 and FIG. 4 is started, for example, when an instruction to start cardiac function evaluation using STE is accepted from the operator. The procedure illustrated in FIG. 4 corresponds to the process at step S105 in FIG. 3. The procedure illustrated in FIG. 3 and FIG. 4 is only an example and embodiments are not limited to the illustration in the drawings.

At step S101, the processing circuitry 170 determines whether it is the process timing. For example, if an instruction to start cardiac function evaluation using STE is accepted from the operator, the processing circuitry 170 determines that it is the process timing (Yes at step S101) and starts the processes at step S102 and subsequent steps. If it is not the process timing (No at step S101), the processes at step S102 and subsequent steps are not started and the processing functions in the processing circuitry 170 are on standby.

If step S101 is positive, at step S102, the transmitting/receiving circuitry 110 performs ultrasound scanning. For example, the transmitting/receiving circuitry 110 causes the ultrasound probe 101 to transmit ultrasound to a two-dimensional scan region (A4C section and A2C section) including the heart (left ventricle) of the subject P and generates reflected wave data from reflected wave signals received by the ultrasound probe 101. The transmitting/receiving circuitry 110 repeats transmission and reception of ultrasound in accordance with a frame rate and successively generates reflected wave data in frames. The B-mode processing circuitry 120 then successively generates B-mode data in frames from the reflected wave data in frames generated by the transmitting/receiving circuitry 110, for each of the A4C section and the A2C section.

At step S103, the image generating circuitry 140 generates time-series ultrasound image data. For example, the image generating circuitry 140 successively generates B-mode image data in frames from the B-mode data in frames generated by the B-mode processing circuitry 120, for each of the A4C section and the A2C section.

In other words, the acquisition function 171 acquires a plurality of pieces of medical image data arranged in time series over at least one cardiac cycle in which a region including the heart of the subject P is imaged, by controlling the processes in the transmitting/receiving circuitry 110, the B-mode processing circuitry 120, and the image generating circuitry 140. The heart is an example of the pulsative target (pulsative part).

At step S104, the tracking function 172 sets a region of interest in the initial phase. For example, the tracking function 172 sets a region of interest at positions corresponding to the inner membrane and the outer membrane of the left ventricle, for each of ultrasound image data of the A4C section and the A2C section in the initial frame.

At step S105, the tracking function 172 performs a tracking process. For example, the tracking function 172 performs a plurality of motion estimation processes using an image correlation at frame intervals different from each other on an identical position for the pieces of medical image data and determines most likely second motion information from among a plurality of pieces of first motion information estimated by the motion estimation processes.

Referring now to FIG. 4, the tracking process at step S105 will be described. Hereinafter a movement vector estimated by the motion estimation process using an image correlation at frame intervals “N” (pattern matching process) is denoted as “V(N)”. The movement vector is an example of “motion information”.

At step S201, the tracking function 172 performs a first motion estimation process using an image correlation at one-frame intervals. More specifically, the tracking function 172 performs the motion estimation process by STE without decimating frames to estimate a movement vector “V(1)”. Any known technology can be applied to the motion estimation process by STE.

At step S202, the tracking function 172 performs a second motion estimation process using an image correlation at two-frame intervals. More specifically, the tracking function 172 performs the motion estimation process by STE while decimating one frame to estimate a movement vector “V(2)”. Any known technology can be applied to the motion estimation process by STE.

At step S203, the tracking function 172 performs a third motion estimation process using an image correlation at three-frame intervals. More specifically, the tracking function 172 performs the motion estimation process by STE while decimating two frames to estimate a movement vector “V(3)”. Any known technology can be applied to the motion estimation process by STE.

At step S204, the tracking function 172 selects a movement vector having the largest velocity component from among a plurality of movement vectors at each position. Specifically, the tracking function 172 selects (determines) “V(N)/N” (movement vector per frame) having the largest “|V(N)/N|” as the actual movement vector, for a plurality of candidate movement vectors “V(N)” estimated at frame intervals “N”. Here, “1×1” is the absolute value of x.

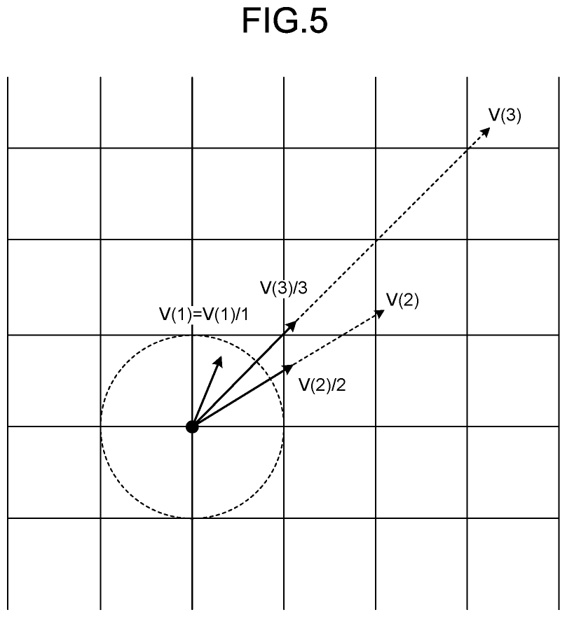

Referring to FIG. 5, the process of the tracking function 172 according to the first embodiment will be described. FIG. 5 is a diagram for explaining the process of the tracking function 172 according to the first embodiment. In the example illustrated in FIG. 5, a movement vector is selected from among three movement vectors “V(1)”, “V(2)”, and “V(3)” estimated for the same position (black circle in the drawing).

As illustrated in FIG. 5, the tracking function 172 calculates “|V(1)/1|”, “|V(2)/2|”, and “|V(3)/3|” from three movement vectors “V(1)”, “V(2)”, and “V(3)”, respectively. The tracking function 172 then compares the calculated values and selects the movement vector “V(3)/3” having the largest velocity component. Since there are movement vectors calculated by decimating frame intervals, it is preferable to calculate a movement vector “V(N)/N” per frame.

In this way, the tracking function 172 selects the most likely movement vector as the actual movement vector, based on the presumption that “the absolute value of a vector is largest when the accuracy is highest”.

At step S205, the tracking function 172 outputs the selected movement vector for each position. In the example in FIG. 5, the tracking function 172 outputs a movement vector “V(N)/N” per frame. The candidate movement vector may be referred to as “first motion information”. The movement vector output by the tracking function 172 is a movement vector actually used as a tracking result and may be referred to as “second motion information”.

The description will return to FIG. 3. At step S106, the calculation function 173 calculates an index value. For example, the calculation function 173 calculates a variety of cardiac function indices from the second motion information calculated for respective ultrasound image data of the A4C section and the A2C section, using the modified-Simpson's method. Examples of the calculated cardiac function indices include volume information such as end diastolic volume (EDV), end systolic volume (ESV), and ejection fraction (EF) of left ventricle (LV) and global longitudinal strain (GLS) information.

Any known technology can be applied to the cardiac function indices calculated by the calculation function 173 and the calculation method therefor. The calculation function 173 can calculate a variety of cardiac function indices when three-dimensional STE is applied, in addition to two-dimensional STE. For example, when three-dimensional STE is applied, the calculation function 173 can also define an area change ratio (AC) on a boundary surface of the inner membrane or the middle layer.

At step S107, the output control function 174 outputs index values. For example, the output control function 174 allows the display 103 to display a variety of cardiac function indices calculated by the calculation function 173. The output control function 174 may output information to the display 103 as well as a storage medium or another information processing apparatus, for example. The output control function 174 may output any image data, in addition to the index values.

The procedure illustrated in FIG. 3 and FIG. 4 is only an example and embodiments are not limited to the illustration in the drawings. For example, the processes at step S201 to step S203 illustrated in FIG. 4 are not necessarily performed in the order illustrated in the drawing but may be performed in different order or may be performed simultaneously.

Although the frame intervals “N” is “1, 2, 3” in FIG. 4, embodiments are not limited thereto. The frame intervals “N” may be a combination of any frame intervals, such as “1, 2” or “2, 4”, as long as different frame intervals are included. However, in order to perform an accurate tracking process, it is preferable that “1” is included and the maximum frame interval is not too wide.

As described above, in the ultrasound diagnostic apparatus 1 according to the first embodiment, the acquisition function 171 acquires a plurality of pieces of medical image data arranged in time series over at least one cardiac cycle in which a region including a pulsative target of a subject is imaged. The tracking function 172 then performs a plurality of motion estimation processes using an image correlation at frame intervals different from each other on an identical position for the pieces of medical image data and determines most likely second motion information from among a plurality of pieces of first motion information estimated by the motion estimation processes. With this process, the ultrasound diagnostic apparatus 1 can improve the accuracy in cardiac function evaluation.

For example, the ultrasound diagnostic apparatus 1 according to the first embodiment performs the process described above, so that a movement vector estimated at short frame intervals is selected in a phase or a position in which deformation or the amount of motion is large and a high frame rate is advantageous, whereas a movement vector estimated at long (decimated) frame intervals is selected in a phase or a position in which the amount of motion is small and a low frame rate is advantageous. Hence, a low-speed movement vector can be detected even at a high frame rate, and the tracking accuracy is improved in any phases. As a result, the possibility that the output values of EF and GLS information are underestimated at a high frame rate is reduced.

In the first embodiment, the most likely movement vector is selected as the actual movement vector, based on the presumption that “the absolute value of a vector is largest when the accuracy is highest”. However, any other selection criteria are possible. For example, a correlation coefficient may be used as the confidence level of movement vectors, and a movement vector “V(N)” with a high confidence level may be selected. However, this is not preferable as a selection criterion because in this case, the shorter the frame interval is, the higher the correlation coefficient is, and in most cases, a movement vector with the smallest frame interval is selected. When a movement vector is obtained by integrating (averaging or weight-averaging) a plurality of movement vectors with different frame intervals, values with low accuracy are included, and consequently, the accuracy tends to deteriorate. Selecting a movement vector having the median vector absolute value (median process) has an effect similar to the averaging process, and the accuracy tends to deteriorate compared to when the maximum is selected. In the first embodiment, therefore, it is preferable to select the most likely movement vector based on the presumption described above.

### First Modification to First Embodiment

A highly accurate movement vector is not necessarily selected in some cases, only by selecting a movement vector based on the presumption that “the absolute value of a vector is largest when the accuracy is highest”.

For example, when the tracking target is deformed, the correlation between signals decreases as the decimated frame intervals increase, and the quality (accuracy) of the estimated motion is generally thought to deteriorate. It is therefore not always preferable that motion information (movement vector) estimated at decimated frame intervals is selected although the amount of motion of the tracking target is sufficiently large. In the present embodiment, it is preferable that motion information estimated at decimated frame intervals is selected “when the amount of motion of the target is sufficiently small under the condition of a high frame rate”. Then, in a first modification to the first embodiment, a process of imposing a restriction such that motion information estimated at decimated frame intervals is not unduly selected, using a determination criterion “when the amount of motion is sufficiently small” will be described.

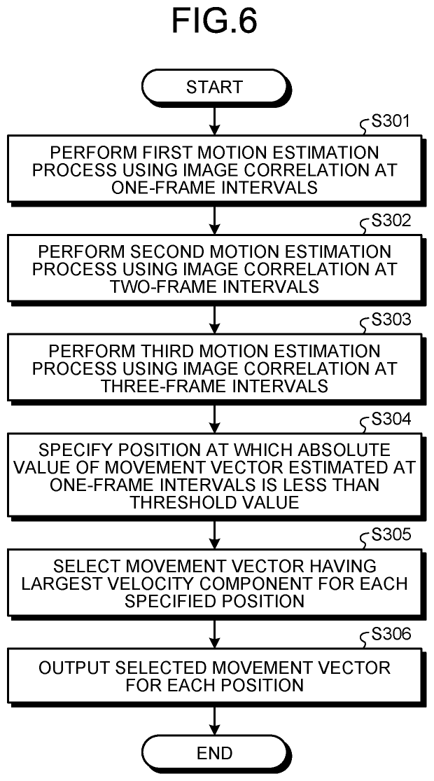

Referring to FIG. 6, a procedure in the ultrasound diagnostic apparatus 1 according to the first modification to the first embodiment will be described. FIG. 6 is a flowchart illustrating a procedure in the ultrasound diagnostic apparatus 1 according to the first modification to the first embodiment. The procedure illustrated in FIG. 6 corresponds to the process at step S105 in FIG. 3. The processes at step S301, 5302, 5303, and 5306 illustrated in FIG. 6 are similar to the processes at step S201, 5202, 5203, and 5205 illustrated in FIG. 4 and will not be further elaborated.

At step S304, the tracking function 172 specifies a position at which the absolute value of the movement vector estimated at one-frame intervals is less than a threshold value. Here, the tracking function 172 uses a value based on the pixel size as the threshold value.

For example, the tracking function 172 compares the magnitude of the absolute value “|V(1)/1|” of motion estimated at one-frame intervals with a threshold “a pixels” at each position and specifies a position with the absolute value less than the threshold value. Here, the threshold value is set to “a pixels” in consideration of the background of motion estimation limited to units of pixels. In a two-dimensional case, “α” is preferably approximately sqrt(2). This is because “α=1” is the smallest motion estimation unit when detection of only motion vectors horizontal (or vertical) to a pixel grid is taken into consideration, but when diagonal motion components are taken into consideration, the smallest estimation unit is sqrt(2). For a similar reason, in a three-dimensional case, “α” is preferably approximately sqrt(3). The description of “approximately” sqrt(2) and “approximately” sqrt(3) is intended not to limit values to exact matches with sqrt(2) and sqrt(3) but to permit values deviated in a range that does not affect the process.

At step S305, the tracking function 172 selects first motion information having the largest velocity component as second motion information, for each specified position. More specifically, when the magnitude of the absolute value “|V(1)/1|” of motion estimated at one-frame intervals is less than the threshold value “a pixels”, the tracking function 172 permits selection of first motion information (N=2 or more) estimated by decimating frame intervals. For a position at which the magnitude of “|V(1)/1|” is equal to or greater than the threshold value, the movement vector “V(1)” is determined as it is as second motion information.

In this way, the tracking function 172 according to the first modification to the first embodiment specifies a position at which the absolute value of first motion information estimated by the motion estimation process using an image correlation at one-frame intervals is less than a threshold value. The tracking function 172 then selects first motion information having the largest velocity component as second motion information, for each specified position. With this process, when the amount of motion of a tracking target is sufficiently large, the ultrasound diagnostic apparatus 1 according to the first modification to the first embodiment prevents motion information estimated at decimated frame intervals from being unduly selected and thereby improves the accuracy in cardiac function evaluation.

### Second Modification to First Embodiment

For example, the maximum value of frame intervals “N” by decimation is preferably determined according to the frame rate, because it is preferable that motion information estimated at decimated frame intervals is selected “when the amount of motion of the target is sufficiently small under the condition of a high frame rate”.

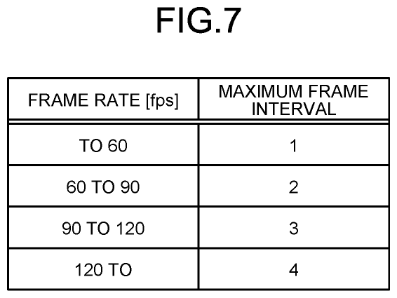

Referring to FIG. 7, a process of the tracking function 172 according to a second modification to the first embodiment will be described. FIG. 7 is a diagram for explaining a process of the tracking function 172 according to the second modification to the first embodiment. FIG. 7 illustrates a table indicating the correspondence between the frame rate and the maximum frame intervals. The table illustrated in FIG. 7 is stored in advance, for example, in a storage device that the tracking function 172 can refer to, such as the internal storage circuitry 160.

In the example illustrated in FIG. 7, in the record on the first row of the table, the frame rate “lower than 60” is stored in association with the maximum frame intervals “1”. This indicates that when the frame rate is lower than 60 fps, decimation is not performed and the motion estimation process using an image correlation at one-frame intervals is performed. In the record on the second row of the table, the frame rate “60 to 90” is stored in association with the maximum frame intervals “2”. This indicates that when the frame rate is 60 fps or higher and lower than 90 fps, the motion estimation process using an image correlation at one-frame intervals and the motion estimation process using an image correlation at two-frame intervals are performed. In the record on the third row of the table, the frame rate “90 to 120” is stored in association with the maximum frame intervals “3”. This indicates that when the frame rate is 90 fps or higher and lower than 120 fps, the motion estimation process using an image correlation at one-frame intervals, the motion estimation process using an image correlation at two-frame intervals, and the motion estimation process using an image correlation at three-frame intervals are performed. In the record on the fourth row of the table, the frame rate “120 or higher” is stored in association with the maximum frame intervals “4”. This indicates that when the frame rate is 120 fps or higher, the motion estimation process using an image correlation at one-frame intervals, the motion estimation process using an image correlation at two-frame intervals, the motion estimation process using an image correlation at three-frame intervals, and the motion estimation process using an image correlation at four-frame intervals are performed.

As a specific example, when the frame rate of medical image data acquired by the acquisition function 171 is “120”, the tracking function 172 refers to the table illustrated in FIG. 7 and determines on the maximum frame intervals “4”. The tracking function 172 then performs the motion estimation process at each of the frame intervals up to the determined maximum frame intervals. Specifically, the tracking function 172 successively or concurrently performs the motion estimation process using an image correlation at one-frame intervals, the motion estimation process using an image correlation at two-frame intervals, the motion estimation process using an image correlation at three-frame intervals, and the motion estimation process using an image correlation at four-frame intervals. In this case, the tracking function 172 calculates four movement vectors “V(1)”, “V(2)”, “V(3)”, and “V(4)” as the first motion information at each position. The tracking function 172 then selects a movement vector having the largest velocity component from among the four movement vectors “V(1)”, “V(2)”, “V(3)”, and “V(4)” estimated at each position.

In this way, the tracking function 172 according to the second modification to the first embodiment determines the maximum value of frame intervals, based on the frame rate of a plurality of pieces of medical image data. The tracking function 172 then performs the motion estimation process at each of the frame intervals up to the determined maximum value. The tracking function 172 then selects one having the largest velocity component as second motion information from among the pieces of first motion information estimated for each position. With this process, the ultrasound diagnostic apparatus 1 according to the second modification to the first embodiment determines an appropriate frame interval according to the frame rate and does not perform the motion estimation process with unnecessary frame decimation, thereby efficiently improving the accuracy in cardiac function evaluation.

### Second Embodiment

In the first embodiment, after a plurality of motion estimation processes using an image correlation at different frame intervals are performed, most likely second motion information is selected from among a plurality of pieces of estimated first motion information. However, embodiments are not limited thereto. For example, first, the amount of motion may be analyzed by performing preliminary tracking (motion estimation process) at one-frame intervals, and main tracking may be performed at frame intervals according to the magnitude of the amount of motion.

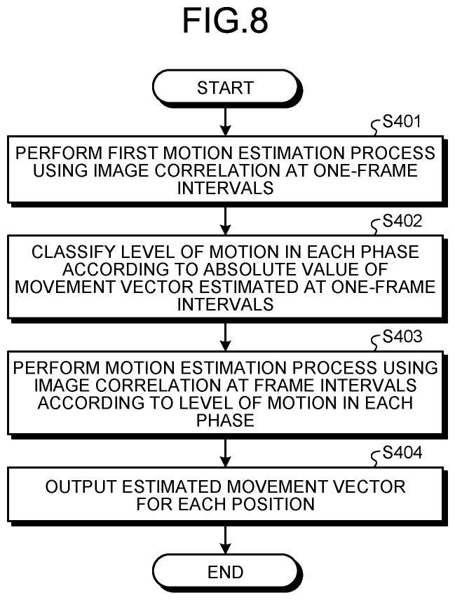

Referring to FIG. 8, a procedure in the ultrasound diagnostic apparatus 1 according to a second embodiment will be described. FIG. 8 is a flowchart illustrating the procedure in the ultrasound diagnostic apparatus 1 according to the second embodiment. The procedure illustrated in FIG. 8 corresponds to the process at step S105 in FIG. 3. The procedure illustrated in FIG. 8 is only an example and embodiments are not limited to the illustration in the drawing.

At step S401, the tracking function 172 performs, as preliminary tracking, a first motion estimation process using an image correlation at one-frame intervals. More specifically, the tracking function 172 performs the motion estimation process by STE without decimating frames to estimate a movement vector “V(1)”. Any known technology can be applied to the motion estimation process by STE.

At step S402, the tracking function 172 classifies the level of motion in each phase, according to the absolute value of the movement vector estimated at one-frame intervals. For example, the tracking function 172 calculates the average amount of motion representing global motion of the left ventricle, using the absolute value of the movement vector at each position estimated by the preliminary tracking.

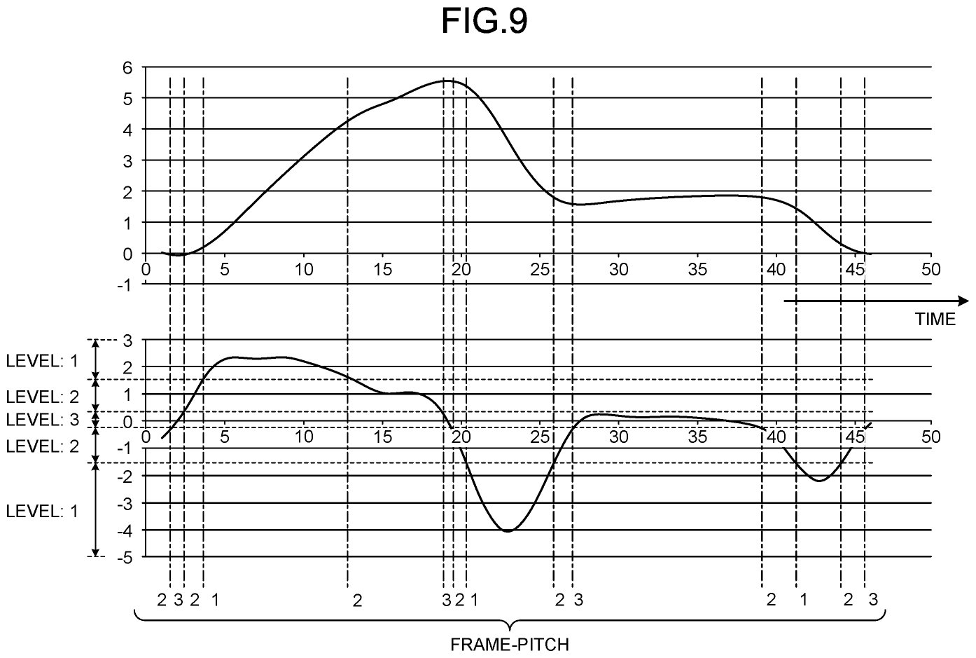

Referring to FIG. 9, the process of the tracking function 172 according to the second embodiment will be described. FIG. 9 is a diagram for explaining the process of the tracking function 172 according to the second embodiment. In the upper section in FIG. 9, the vertical axis corresponds to global displacement [mm] of the left ventricle wall and the horizontal axis corresponds to time (frame). In the lower section in FIG. 9, the vertical axis corresponds to global motion [cm/sec] of the left ventricle wall and the horizontal axis corresponds to time (frame). The horizontal axes (time axes) in the upper section in FIG. 9 and the lower section in FIG. 9 correspond to each other.

As illustrated in FIG. 9, the tracking function 172 classifies the phases into three stages of levels “1” to “3”, according to the absolute value of motion illustrated in the lower section in FIG. 9. Here, level “1” corresponds to motion of 1.5 [cm/sec] or more, level “2” corresponds to motion of 0.5 [cm/sec] or more and less than 1.5 [cm/sec], and level “3” corresponds to motion of less than 0.5 [cm/sec].

In the example illustrated in FIG. 9, the cardiac phases s′ that is the systolic peak phase, e′ that is the early diastolic peak phase, and a′ that is atrial systolic phase are classified into level “1” representing fast motion, and the cardiac phases with no motion and almost at a standstill are classified into level “3”. In this way, the tracking function 172 classifies levels in units of image data in each frame.

At step S403, the tracking function 172 performs, as main tracking, the motion estimation process using an image correlation at frame intervals (frame pitches) according to the level of motion in each phase. In the example in FIG. 9, the tracking function 172 performs the motion estimation process at one-frame intervals in a phase of level “1”, at two-frame intervals in a phase of level “2”, and at three-frame intervals in a phase of level “3”. Since the phase of level “1” has one-frame intervals, the tracking result (movement vector) in the preliminary tracking can be applied.

At step S404, the tracking function 172 outputs the movement vector estimated by the main tracking, for each position. The movement vector “V(N)” estimated by the motion estimation process performed at intervals of two or more frames is converted into a movement vector “V(N)/N” per frame before being output.

The description given with reference to FIG. 8 and FIG. 9 is only an example and embodiments are not limited to the illustration in the drawings. For example, in FIG. 8, the first motion estimation process serving as preliminary tracking is performed at one-frame intervals. However, it may be performed at intervals of any number of frames.

In FIG. 9, the levels are classified into three stages. However, the levels can be classified into any number of stages. Furthermore, the amount of motion that defines each level is not limited to the values illustrated in the drawing but may be set to any value.

In FIG. 9, the levels of motion are classified in units of image data in each frame, for simplicity of the process. However, embodiments are not limited thereto. For example, the tracking function 172 may classify the levels in units of local regions or in units of pixels of image data in each frame. When the levels are classified in units of local regions, the tracking function 172 calculates the average amount of motion representing the motion of a local region of the left ventricle and classifies the level according to the absolute value of motion for each local region. When the levels are classified in units of pixels, the tracking function 172 calculates the amount of motion of each pixel and classifies the level according to the absolute value of motion for each pixel.

As described above, in the ultrasound diagnostic apparatus 1 according to the second embodiment, the tracking function 172 estimates first motion information by performing the motion estimation process using an image correlation at first frame intervals. Subsequently, the tracking function 172 classifies the degree of motion in each phase, according to the magnitude of the first motion information estimated at the first frame intervals. The tracking function 172 then estimates second motion information by performing the motion estimation process at second frame intervals according to the degree of motion in each phase. With this process, the ultrasound diagnostic apparatus 1 according to the second embodiment can improve the accuracy in cardiac function evaluation while suppressing increase in process load due to the motion estimation process.

The process of the tracking function 172 according to the second embodiment can be combined with the processes described in the first modification and the second modification to the first embodiment. For example, when the process is combined with the first modification to the first embodiment, it is preferable that the tracking function 172 permits selection of first motion information (N=2 or more) estimated by decimating frame intervals when the magnitude of the absolute value “|V(1)/1|” of the motion estimated at one-frame intervals is less than the threshold value “a pixels”.

When the process is combined with the second modification to the first embodiment, it is preferable that the tracking function 172 determines the maximum value of frame intervals, that is, the maximum value of the level of motion, based on the frame rate of a plurality of pieces of medical image data. For example, when the maximum value of frame intervals is “3”, the tracking function 172 sets the maximum frame intervals defined by the level of motion to “3”. When the maximum value of frame intervals is “4”, the tracking function 172 sets the maximum frame intervals defined by the level of motion to “4”.

### Other Embodiments

A variety of different modes other than the foregoing embodiments may be carried out.

Application to Medical Image Data Other Than Ultrasound Image Data

For example, in the foregoing embodiments, ultrasound image data captured by the ultrasound diagnostic apparatus 1 is used as medical image data. However, embodiments are not limited thereto. For example, the present embodiment can use, as a process target, medical image data captured by other medical image diagnostic apparatuses, such as computed tomography (CT) image data captured by an X-ray CT apparatus or MR image data captured by a magnetic resonance imaging (MRI) apparatus.

Medical Image Processing Apparatus

For example, in the foregoing embodiments, the processing functions according to embodiments are applied to the ultrasound diagnostic apparatus 1. However, embodiments are not limited thereto. For example, a variety of processing functions for performing a setting process in a three-dimensional coordinate system can also be applied to a medical image processing apparatus.

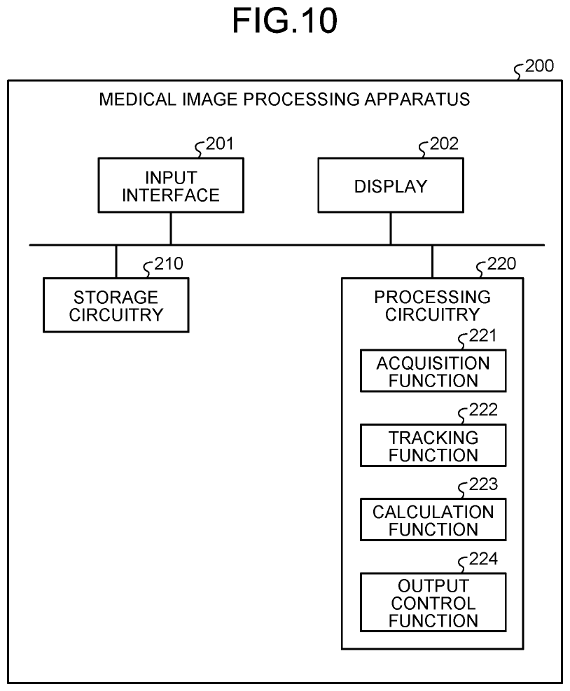

Referring to FIG. 10, a configuration of a medical image processing apparatus 200 according to other embodiments will be described. FIG. 10 is a block diagram illustrating a configuration example of the medical image processing apparatus 200 according to other embodiments.

As illustrated in FIG. 10, the medical image processing apparatus 200 includes an input interface 201, a display 202, storage circuitry 210, and processing circuitry 220. The input interface 201, the display 202, the storage circuitry 210, and the processing circuitry 220 are connected to communicate with each other. A plurality of pieces of medical image data captured by any medical image diagnostic apparatus are stored in advance in the storage circuitry 210.

The processing circuitry 220 performs an acquisition function 221, a tracking function 222, a calculation function 223, and an output control function 224. Here, the processing functions including the acquisition function 221, the tracking function 222, the calculation function 223, and the output control function 224 can perform processes similar to the processing functions including the acquisition function 171, the tracking function 172, the calculation function 173, and the output control function 174 illustrated in FIG. 1.

More specifically, in the medical image processing apparatus 200, the acquisition function 221 acquires a plurality of pieces of medical image data arranged in time series over at least one cardiac cycle in which a region including a pulsative target of a subject is imaged. For example, the acquisition function 221 acquires a plurality of pieces of medical image data by reading a plurality of pieces of medical image data from the storage circuitry 210. The tracking function 222 then performs a plurality of motion estimation processes using an image correlation at frame intervals different from each other on an identical position for the pieces of medical image data and determines most likely second motion information from among a plurality of pieces of first motion information estimated by the motion estimation processes. With this process, the medical image processing apparatus 200 can improve the accuracy in cardiac function evaluation.

The constituent elements in each apparatus illustrated in the drawings are functional and conceptual and are not necessarily physically configured as illustrated in the drawings. More specifically, the specific manner of distribution and integration in each apparatus is not limited to the one illustrated in the drawings, and the whole or a part of the apparatus may be configured so as to be functionally or physically distributed or integrated in any units, depending on load and use conditions. The processing functions performed in each apparatus may be entirely or partially implemented by a CPU and a computer program analyzed and executed by the CPU or may be implemented by hardware using wired logic.

Among the processes described in the foregoing embodiments and modifications, all or some of the processes automatically performed may be performed manually, or all or some of the processes performed manually may be performed automatically using a known method. Furthermore, the procedure, the control procedure, the specific names, and information including a variety of data and parameters described in the document and illustrated in the drawings can be changed as appropriate unless otherwise specified.

The medical image processing method described in the foregoing embodiments and modifications can be implemented by executing a medical image processing program prepared in advance in a computer such as a personal computer or a workstation. The medical image processing program can be distributed over a network such as the Internet. The medical image processing program may be recorded in a computer-readable non-transitory recording medium, such as a hard disk, a flexible disk (FD), a CD-ROM, an MO, or a DVD, and read from the recording medium and executed by a computer.

According to at least one embodiment described above, the accuracy in cardiac function evaluation can be improved.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound diagnostic apparatus comprising processing circuitry configured to
acquire a plurality of pieces of medical image data arranged in time series over at least one cardiac cycle in which a region including a pulsative target of a subject is imaged, and
perform a plurality of motion estimation processes using a pattern matching at frame intervals different from each other on an identical position for the pieces of medical image data and determine most likely second motion information from among a plurality of pieces of first motion information estimated by the motion estimation processes.

2. The ultrasound diagnostic apparatus according to claim 1, wherein the processing circuitry selects first motion information having a largest velocity component as the second motion information from among the pieces of first motion information.

3. The ultrasound diagnostic apparatus according to claim 1, wherein
the processing circuitry is configured to
estimate the first motion information by performing a motion estimation process using the pattern matching at first frame intervals,
classify a degree of motion in each phase, according to a magnitude of the first motion information estimated at the first frame intervals, and
estimate the second motion information by performing a motion estimation process at frame intervals according to the degree of motion in each phase.

4. The ultrasound diagnostic apparatus according to claim 1, wherein the processing circuitry determines a maximum value of the frame intervals, based on a frame rate of the pieces of medical image data.

5. The ultrasound diagnostic apparatus according to claim 1, wherein
the processing circuitry is configured to
specify a position at which an absolute value of first motion information estimated by the motion estimation process using the pattern matching at one-frame intervals is less than a threshold value, and
select first motion information having a largest velocity component as the second motion information for each specified position.

6. The ultrasound diagnostic apparatus according to claim 5, wherein the processing circuitry uses a value based on a pixel size as the threshold value.

7. A medical image processing apparatus comprising processing circuitry configured to
acquire a plurality of pieces of medical image data arranged in time series over at least one cardiac cycle in which a region including a pulsative target of a subject is imaged, and
perform a plurality of motion estimation processes using a pattern matching at frame intervals different from each other on an identical position for the pieces of medical image data and determine most likely second motion information from among a plurality of pieces of first motion information estimated by the motion estimation processes.

8. A medical image processing method comprising:
acquiring a plurality of pieces of medical image data arranged in time series over at least one cardiac cycle in which a region including a pulsative target of a subject is imaged; and
performing a plurality of motion estimation processes using a pattern matching at frame intervals different from each other on an identical position for the pieces of medical image data and determining most likely second motion information from among a plurality of pieces of first motion information estimated by the motion estimation processes.
